(19) United States
(12) Patent Application Publication
(10) Pub. No.: US 2026/0130684 A1
(43) Pub. Date: May 14, 2026

(54) DEVICES, SYSTEMS, AND METHODS FOR DELIVERING MEDICAL INSTRUMENTS WITHIN A PATIENT

(71) Applicant: BOSTON SCIENTIFIC SCIMED, INC., Maple Grove, MN (US)

(72) Inventors: Jason N. Mehta, Austin, TX (US); Ryan Davis Gilkey, Austin, TX (US); Alexander R. Cohen, Austin, TX (US); Timothy Noah Foster-Frau, Austin, TX (US); John Mims, Austin, TX (US)

(73) Assignee: BOSTON SCIENTIFIC SCIMED, INC., Maple Grove, MN (US)

(21) Appl. No.: 19/382,735

(22) Filed: Nov. 7, 2025

Related U.S. Application Data
(60) Provisional application No. 63/718,221, filed on Nov. 8, 2024.

(51) Int. Cl. A61B 17/32; A61B 17/00

(52) U.S. Cl. CPC A61B 17/320016; A61B 2017/0034

(57) ABSTRACT

An auxiliary device configured to be positioned alongside an elongate delivery member for a medical instrument, such as a medical scope. The auxiliary device is configured to deliver one or more medical instruments independently of a medical instrument delivered by the elongate delivery member. The distal ends of the auxiliary device and the elongate delivery member may be delivered into a patient's body in a configuration adjacent each other, and may be separated from each other to allow separate and independent operation of medical instruments respectively delivered by the auxiliary device and the elongate delivery member.

### CROSS-REFERENCE TO RELATED APPLICATIONS

This application claims the benefit of priority under 35 U.S.C. § 119 to U.S. Provisional Application No. 63/718,221, filed Nov. 8, 2024, the entire disclosure of which is hereby incorporated by reference herein for all purposes.

### FIELD

The present disclosure relates generally to the field of medical devices which deliver medical instruments for performing a procedure within a patient's body. More particularly, the present disclosure relates to devices, systems, and methods which provide a second, auxiliary delivery device for a medical instrument along a first delivery device for a medical instrument, each delivery device capable of delivering separate medical instruments.

### BACKGROUND

Minimally-invasive procedures, such as transluminal, transcatheter, percutaneous, endoscopic, etc., procedures, have various benefits over open surgery. Whereas open surgery requires cutting open the patient's body to gain access into the patient's body and internal anatomical structures, minimally-invasive procedures access a target site within the patient via a natural orifice (or, in some instances, a small incision not considered to constitute an open-surgery cut). Such methods induce little to no bleeding (minimized to no surgical cutting), require minimal if any implements to hold open the access opening, reduce various risks typical of open surgery, reduce recovery times, and have various other benefits over open surgery. Despite the various benefits over open surgery, the limited available working space within a patient for performing minimally-invasive procedures presents various challenges. Often more than one medical instrument, tool, device, etc., is used during a given medical procedure. However, the lumen of a delivery device for such medical instruments, tools, devices, etc., often does not accommodate more than one medical instrument, tool, device, etc., to be extended therethrough. Therefore, multiple device exchanges may be required during the procedure. Moreover, the maneuverability of the instruments at the target site for the procedure may be limited by the delivery device through which the instrument extends. During Endo Luminal Surgery (ESL) procedures/endoscopic surgical procedures, there may be a need to add additional channels to a medical scope (e.g., an endoscope) to deploy multiple tools. However, maneuverability and control of the multiple channels and or devices may present a challenge, particularly if different movements and/or actions are to be performed by the different devices. For instance, during Endoscopic Submucosal Dissection (ESD), a physician may want to apply tissue traction to help visualize the separation being performed between the mucosa/submucosa and the muscle/muscularis. In this instance, there generally is a need to deploy a tool or device to apply traction, and then to make that tool or device independent of the movement directions of the endoscope (through which a tissue-separating device is extended). However, typical deployment of a traction device through the working channel of the endoscope limits the movement and operation of the traction device to being affected by, and not being independent of, movements of the endoscope. As may be appreciated, the limited spaces in which minimally-invasive procedures typically are performed also impacts the maneuverability of various accessories, such as traction devices, within the body. Accordingly, there remains a need for improved devices, systems, and associated methods to deliver and use a medical device independently of another medical device, such as a medical scope, within a patient.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system, for delivering medical instruments into a patient's body, includes a primary elongate member having a flexible tubular elongate member with a proximal portion proximal to a transition point along the flexible tubular elongate member, and an increased-flexibility section distal to the transition point and more flexible than the proximal portion; and an auxiliary elongate member having an external elongate member with a proximal portion, and a distal increased-flexibility section extending distally from the proximal portion. In some aspects, the auxiliary elongate member is configured to extend along the primary elongate member and to be coupled adjacent to the primary elongate member to be moved by the primary elongate member; and the distal increased flexibility section of the auxiliary elongate member is configured to be operably coupled with and to move with the increased-flexibility section of said primary elongate member and has sufficient structural stability to remain in a position determined by the primary elongate member before the primary elongate member is moved away from the auxiliary elongate member.

In some aspects, the system further includes a return element coupling the primary elongate member and the auxiliary elongate member. In some aspects, the return element movably couples the distal increased flexibility section of the auxiliary elongate member with respect to the increased flexibility section of the primary elongate member. In some aspects, the return element is translatable to adjust the relative positions of the distal increased flexibility section of the auxiliary elongate member and the increased flexibility section of the primary elongate member. In some aspects, the return element is controllable to adjust the relative positions of the distal increased flexibility section of the auxiliary elongate member and the increased flexibility section of the primary elongate member.

In some aspects, the proximal portion of the auxiliary elongate member is configured to be coupled with the proximal portion of the primary elongate member proximal to the transition point along the primary elongate member.

In some aspects, the proximal portion of the auxiliary elongate member has a distal end coupled to the flexible tubular elongate member of the primary elongate member along the transition point.

In some aspects, the proximal portion of the auxiliary elongate member is a sheath configured to receive an exterior of the primary elongate member. In some aspects, the sheath defines a lumen for longitudinal passage of a medical instrument therethrough.

In some aspects, the system further includes a flexible tubular elongate member extending through the lumen of the sheath and through the distal increased-flexibility portion of the auxiliary elongate member, and defining a tool lumen for longitudinal passage of a medical instrument therethrough.

In some aspects, the distal increased-flexibility section of the auxiliary elongate member is formed with a plurality of spaced apart segments. In some aspects, the plurality of spaced apart segments are coupled together with control wires imparting structural stability to the distal increased-flexibility section.

In some aspects, the system further includes a medical instrument longitudinally translatable through a lumen defined through said auxiliary elongate member. In some aspects, the medical instrument maintains a position of the distal increased-flexibility section of the auxiliary elongate member before the increased-flexibility section of the primary elongate member is moved away from the distal increased-flexibility section of the auxiliary elongate member. In some aspects, the system further includes a flexible tubular elongate member extending through the auxiliary elongate member and defining a tool lumen for longitudinal passage of the medical instrument therethrough. In some aspects, the flexible tubular elongate member extends through the distal increased-flexibility portion of the auxiliary elongate member.

In accordance with various principles of the present disclosure, a system, for delivering medical instruments into a patient's body, includes a flexible external elongate member having a proximal portion and a distal increased-flexibility section extending distally from the proximal portion; and a medical instrument longitudinally translatable through a lumen defined through the flexible external elongate member. In some aspects, the proximal portion of the flexible external elongate member is configured to be coupled with a proximal portion of a flexible steerable elongate member proximal to a transition point along the flexible steerable elongate member; the distal increased-flexibility section of the flexible external elongate member is configured to be operably coupled with and to move with a distal increased-flexibility section of a flexible steerable elongate member defined distal to the transition point; and the medical instrument maintains a position of the distal increased-flexibility section of the flexible external elongate member determined by the flexible steerable elongate member before the increased-flexibility section of the flexible steerable elongate member is moved away from the distal increased-flexibility section of the flexible external elongate member.

In some aspects, the system further includes a return element configured to couple the flexible external elongate member with the primary elongate member. In some aspects, the return element movably couples the distal increased flexibility section of the flexible external elongate member with respect to the increased flexibility section of the primary elongate member. In some aspects, the return element is controllable to adjust the relative positions of the distal increased flexibility section of the flexible external elongate member and the increased flexibility section of the primary elongate member.

In some aspects, the system further includes a flexible tubular elongate member extending through the flexible external elongate member and defining a tool lumen for longitudinal passage of the medical instrument therethrough.

In accordance with various principles of the present disclosure, a method of delivering and operating separate medical instruments includes delivering a first medical instrument with a primary elongate member; delivering a second medical instrument with an auxiliary elongate member extending along the primary elongate member; manipulating a flexible distal end of the primary elongate member, including causing a flexible distal end of the auxiliary elongate member to be similarly manipulated; and separating the flexible distal end of the primary elongate member from the flexible distal end of the auxiliary elongate member to manipulate the flexible distal end of the primary elongate member without also manipulating the flexible distal end of the auxiliary elongate member.

In some aspects, separating the flexible distal end of the primary elongate member from the flexible distal end of the auxiliary elongate member leaves the flexible distal end of the auxiliary elongate member in the configuration of the primary elongate member prior to separation from the auxiliary elongate member.

In some aspects, the method further includes returning the flexible distal end of the primary elongate member to a position adjacent the flexible distal end of the auxiliary elongate member.

In some aspects, the method further includes controlling the relative positions of the flexible distal end of the primary elongate member and the flexible distal end of the auxiliary elongate member by longitudinally translating a return element coupled therebetween. In some aspects, the method further includes manipulating the return element to return the flexible distal end of the primary elongate member to a position adjacent the flexible distal end of the auxiliary elongate member.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of a system, formed in accordance with aspects of the present disclosure, for delivering medical devices within a patient.

FIG. 2 illustrates a system such as illustrated in FIG. 1 in use in a schematic illustration of a treatment site within a patient.

FIG. 3 illustrates a further use position of the system illustrated in FIG. 2.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

The present disclosure describes various medical devices and systems which may be delivered into a patient's body via minimally invasive techniques. More particularly, the present disclosure describes devices and systems, and associated methods, for delivering more than one medical device in the limited space in which minimally invasive procedures typically are performed. It will be appreciated that terms such as devices, instruments, tools, components, accessories, etc., may be used interchangeably herein without intent to limit unless otherwise indicated. The devices, systems, and methods of present disclosure further allow for independent movement of the medical devices which are delivered to a treatment site within the patient's body. It will be appreciated that terms such as target site, target area, target tissue site, target tissue area, target area of tissue, target treatment area, treatment area, target treatment site, treatment site, etc., may be used interchangeably herein, without intent to limit, to refer to an area or region of tissue at which a procedure is to be performed. The term target tissue (and other variations thereof, such as treatment tissue) is used herein to refer to the tissue with respect to which a procedure is to be performed or which is to be treated or otherwise operated on or affected by the devices and/or systems and/or methods disclosed herein. Thus, the target site may be understood as an area or region extending outwardly from or around or surrounding the target tissue (specific tissue in the target tissue area), such as the region a medical professional would consider a working area around the target tissue for performing a procedure with respect to the target tissue.

Minimally invasive procedures are becoming more desirable for removing or repairing target tissue within or in the area of a target site within a patient's body. In contrast with open surgery, minimally invasive surgery involves advancing a medical device into a patient's body through a natural orifice (or small incision, if necessary) and navigated the medical device within the body, such as through an internal body passage, to a target site. Typically, an elongate member is used in minimally-invasive medical procedures to deliver a medical device to a target site for use in performing a procedure. The elongate member may be any suitable size, cross-sectional shape or area, and/or configuration permitting introduction and passage of medical instruments into the patient. Typically, the elongate member is sufficiently flexible to navigate through curved and/or tortuous body passages, particularly if inserted transluminally. In some aspects, the elongate member is a shaft (substantially solid, or hollow and defining a lumen longitudinally therethrough) with a medical device at a distal end thereof. In some aspects, the elongate member is tubular, such as in the form of a catheter, sheath, tube, cannula, etc. (such terms being used interchangeably herein without intent to limit), and defines a lumen through which a medical device is distally advanced to a target site. A tubular elongate member may be used as a delivery device, introducer, etc. A tubular elongate member may or may not have a medical device mounted or otherwise coupled to a distal end thereof. In some aspects, it may be advantageous to deliver a medical device through a delivery device in the form of a tubular elongate member to protect or shield the passage through which the medical device is delivered (e.g., body passage or otherwise) against adverse interactions (e.g., catching, scratching, and/or otherwise) by the medical device delivered therethrough. Additional overtubes, sheaths, etc., may be provided as desired or as necessary to reduce friction or interference of the exterior of any elements with another element.

In addition to the above-described elongate members, various medical devices, such as medical scopes (e.g., endoscopes, arthroscopes, bronchoscopes, colonoscopes, cystoscopes, duodenoscopes, gastroscopes, hysteroscopes, laparoscopes, ureteroscopes, etc.), include an elongate member for insertion into a body passageway or cavity to enable a medical professional to deliver a medical device to a target site within a patient's body to perform minimally-invasive surgical procedures at the target site internal to the patient's body. Medical scopes typically have a proximal end (typically with a control handle) that remains external to the patient, and a distally extending flexible elongate member configured for insertion into the patient. The distally-extending flexible elongate member is generally tubular, and may be known as an insertion tube (and may referenced herein as such for the sake of convenience and without intent to limit). The distally-extending flexible elongate member of the medical scope is inserted into a body cavity or passage of the patient, and is navigated within the patient to an internal treatment site. In general, the distally-extending flexible elongate member of the medical scope has one or more working channels extending longitudinally therethrough through which a medical device may be advanced or retracted. The distally-extending flexible elongate member of the medical scope may also be equipped with one or more accessories such as, for example, a miniature viewing device (optical component, such as a camera), an illumination device (e.g., an LED or optical fiber), lumens for suction, lumens for inflation/irrigation mediums, etc. The distally-extending flexible elongate members of most medical scopes generally have a limited number of working channels, and the outer diameter of such devices generally is limited by the size of the body passage through which the device is to be navigated.

For purposes of the present disclosure, an elongate member with a medical device at a distal end thereof, or a tubular elongate member through which another medical device may be delivered to a target site, or the distally-extending flexible elongate member of a medical scope, or another form of an elongate member may be considered herein to be a delivery device, or, more generally an “elongate member” which delivers a medical instrument into a patient's body and with which systems, devices, and methods of the present disclosure may be used. For the sake of convenience, reference is made herein simply to an elongate member. It should be appreciated that such elongate member may have a medical device mounted or coupled to a distal end thereof (e.g., as an integral device), or may be a tubular elongate member with a medical device extended therethrough. The elongate member may be any suitable size, cross-sectional shape or area, and/or configuration permitting introduction and passage of devices or instruments to the distal end of the elongate member, such as for passage and introduction of medical instruments to a target tissue site. It will be appreciated that the elongate member need not define a lumen therethrough (need not be tubular for passage of a medical device therethrough), but, instead, may include a shaft with a medical device mounted thereon (e.g., on the distal end thereof). Typically, the elongate member has at least a distal portion which is elongate and flexible to be able to be navigated through passages within a patient's body which may be curved and/or tortuous, with a proximal portion extending proximally (out of the patient's body or coupled with a control handle outside the patient's body) to be controlled (maneuvered, or one or more functions thereof, such as camera, irrigation, suction, light, etc., actuated) by a medical professional.

In some aspects, an elongate member may not be sized to readily deliver more than one medical instrument (e.g., may have a lumen with a limited size). In accordance with various principles of the present disclosure, an auxiliary elongate member is provided in addition to an elongate member such as described above. For the sake of differentiating the elongate members, the first elongate member may be referenced as the first or primary elongate member, and the second elongate member may be referenced as an additional or auxiliary elongate member. The auxiliary elongate member provides one or more devices in addition to devices delivered by the primary elongate member. Optionally, the auxiliary elongate member provides one or more channels, in addition to one or more channels which may be provided by the primary elongate member, through which the one or more of the additional devices may be delivered. In some aspects, the auxiliary elongate member may be considered a “sidecar” to the primary elongate member providing additional devices, features, functionalities, etc., to the primary elongate member.

In accordance with various principles of the present disclosure, the auxiliary elongate member allows for delivery of additional/auxiliary medical devices while a medical device is delivered by the primary elongate member. In some aspects, it is desirable for the movement directions and/or operation of the primary elongate member to be independent of the auxiliary elongate member. In accordance with various principles of the present disclosure, at least a portion of the auxiliary elongate member is separable from the primary elongate member, and therefore may allow spatially independent operation of medical devices delivered by the auxiliary elongate member and medical devices delivered by the primary elongate member. As may be appreciated, an auxiliary elongate member formed in accordance with various principles of the present disclosure thereby provides greater flexibility to the medical professional in the use of medical devices and the operation of such devices with respect to tissue at a target site.

In some aspects, the auxiliary elongate member is coupled to the primary elongate member to be delivered together to a treatment site, and is separable from the primary elongate member at the treatment site. In some aspects, the auxiliary elongate member is moved by the primary elongate member, and, once separated, remains generally in the configuration and position of the primary elongate member prior to separation from the auxiliary elongate member. In some aspects, the auxiliary elongate member may be returned to a position adjacent the primary elongate member after separation from the primary elongate member.

Various embodiments of elongate members auxiliary to and movable with a primary elongate member, yet separable from the primary elongate member, and associated systems and methods, will now be described with reference to examples illustrated in the accompanying drawings. Reference in this specification to “one embodiment,” “an embodiment,” “some embodiments”, “other embodiments”, etc., indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

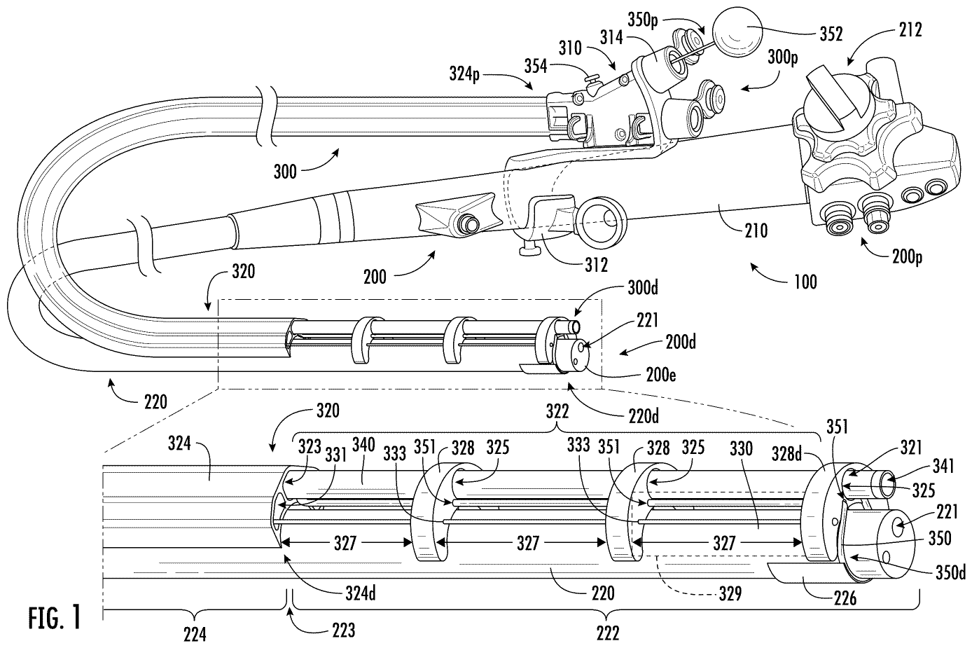

Turning now to the drawings, an example of an embodiment of a system 100 formed in accordance with various principles of the present disclosure for performing procedures within a patient's body is illustrated in FIG. 1. The system includes a primary elongate member illustrated as a medical scope 200, and an auxiliary elongate member 300 extending along the medical scope 200. In some aspects, the auxiliary device 300 may be considered a “sidecar” to the medical scope 200, providing functionality and/or features, and/or delivering another medical instrument usable in conjunction with the medical scope 200. It will be appreciated that suitable configurations of an elongate member other than a medical scope 200 may be utilized, the present disclosure not being limited in this regard.

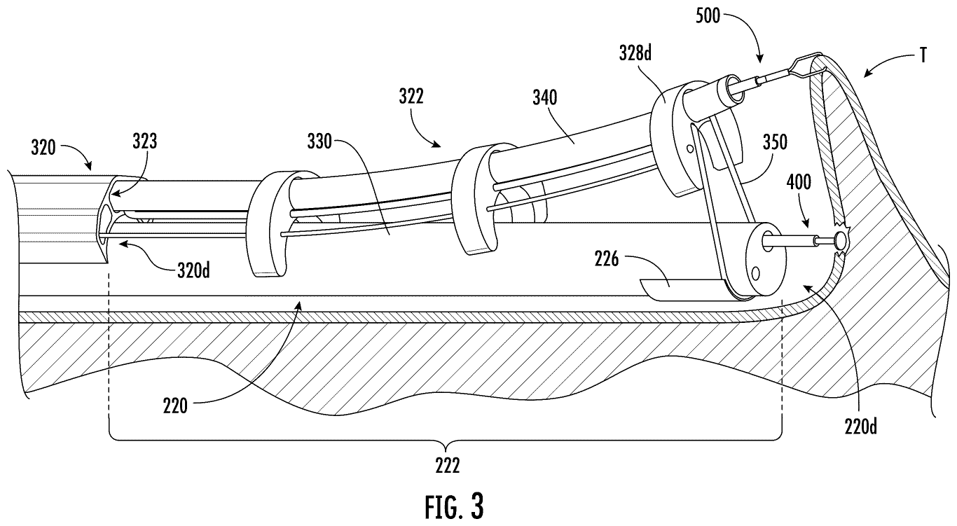

The illustrated example of a medical scope 200 has a proximal end 200p with a control handle 210, and a flexible tubular elongate member 220 (also known as a tubular shaft or insertion member of the medical scope) extending distally from the control handle 210 toward the distal end 200d of the medical scope 200. The flexible tubular elongate member 220 has a distal end 220d which is insertable into a patient to be advanced to a target area within a patient's body in a manner known to those of ordinary skill in the art. The flexible tubular elongate member 220 defines one or more lumens or working channels 221 through which one or more medical tools (e.g., a medical tool 400 such as a cutting instrument as illustrated in FIG. 3) may be extended and delivered to the target area. The distal end 220d of the flexible tubular elongate member 220 typically has an increased-flexibility section 222 more flexible than the proximal portion 224 of the flexible tubular elongate member 220 proximal to the increased-flexibility section 222. The increased-flexibility section 222 may be considered as the portion of the flexible tubular elongate member 220 distal to a transition point 223, and the proximal portion 224 of the flexible tubular elongate member 220 may be considered as the portion of the flexible tubular elongate member 220 proximal to the transition point 223. The control handle 210 may include one or more control knobs 212 for controlling movement of the increased-flexibility section 222 of the flexible tubular elongate member 220, such as to direct the distalmost end 220e of the flexible tubular elongate member 220 toward a selected location (e.g., to direct a viewing element, light, working channel, etc., of the elongate member to such location), in a manner known to those of ordinary skill in the art. For instance, small movements of the distalmost end 220e of the flexible tubular elongate member 220 may be needed to control movement of an instrument extended through the working channel 221 and out the distalmost end 220e of the flexible tubular elongate member 220.

The auxiliary elongate member 300 includes, at a proximal end 300p thereof, a control handle 310 to facilitate manipulation and control of components of the auxiliary elongate member 300 (as described in further detail below). The control handle 310 optionally includes a handle bracket 312 configured for operable engagement with the control handle 210 of the medical scope 200, such as to mount the control handle 310 with respect to the control handle 210 to facilitate access to both in close proximity. The auxiliary elongate member 300 further includes an external elongate member 320 extending distally along the exterior of the flexible tubular elongate member 220 of the medical scope 200 toward the distal end 300d of the auxiliary elongate member 300. In some aspects, the external elongate member 320 is coupled to the flexible tubular elongate member 220 with an external coupler, such as a band, tie, strap, tape, etc. The external elongate member 320 may be in the form of a sheath configured to be inserted into a patient's body alongside the flexible tubular elongate member 220. In some aspects, at least a portion of the external elongate member 320 is shaped to complement or correspond to the exterior of the flexible tubular elongate member 220, such as to receive the flexible tubular elongate member 220 therein, as may be appreciated with reference to the detail view in FIG. 1. The external elongate member 320 defines a tool lumen 321 therethrough, which may be considered to define an additional or auxiliary lumen for use with the medical scope 200. For instance, a medical instrument in addition to a medical instrument delivered by (e.g., through the working channel 221 of) the medical scope 200 may be delivered through the tool lumen 321 of the external elongate member 320 to a treatment site.

In accordance with various principles of the present disclosure, the external elongate member 320 includes a distal increased-flexibility section 322 extending distally from a distal end 324d of a proximal portion 324 of the external elongate member 320. Similar to the increased-flexibility section 222 and the proximal portion 224 of the flexible tubular elongate member 220, the flexibility of the distal increased-flexibility section 322 is greater than the flexibility of the proximal portion 324. In some aspects, the external elongate member 320 is coupled with/mounted on the flexible tubular elongate member 220 with the distal end 324d of the proximal portion 324 adjacent and/or longitudinally aligned with the transition point 223 along the flexible tubular elongate member 220. Such positioning of the distal end 324d of the proximal portion 324 may longitudinally align the distal increased-flexibility section 322 of the external elongate member 320 is with the increased-flexibility section 222 of the flexible tubular elongate member 220. Thus, the distal increased-flexibility section 322 of the external elongate member 320 may move and flex with movements and flexion of the increased-flexibility section 222 of the flexible tubular elongate member 220.

In some aspects, the distal increased-flexibility section 322 is structurally distinct from the proximal portion 324 of the external elongate member 320, such as to be more flexible than the proximal portion 324. For instance, in the example of an embodiment illustrated in FIGS. 1-3, the distal increased-flexibility section 322 is formed from a plurality of axially spaced apart linkages or joints or segments 328 (referenced herein as segments 328 for the sake of convenience and without intent to limit). The segments 328 may be spaced apart by gaps 327 which impart varying rigidity along the length of the distal increased-flexibility section 322, with relatively rigid points, created by the segments 328, separated by less rigid extents, created by the gaps 327. In some aspects, a flexible material 329 is provided between the more rigid segments 328 to provide structural stability without impeding the flexibility of the distal increased-flexibility section 322. For instance, the flexible material 329 may be a silicone with sufficient flexibility to allow the distal increased-flexibility section 322 to follow the movement of the increased-flexibility section 222 of the flexible tubular elongate member 220. The flexible material 329 may be overmolded along at least a portion (or the entire length) of some or all of the segments 328. In some aspects, the edges of the segments 328 are softened so as not to present a sharp and/or rigid end which may contact tissue within the patient's body. Additionally or alternatively, a flexible coating may be provided over at least the edges of the segments 328, such as to soften the edges.

The segments 328 may be coupled together by one or more cables 330 extending through corresponding cable lumens 331 defined axially through the external elongate member 320 and corresponding cable apertures 333 defined through the segments 328. The cables 330 are sufficiently flexible to allow flexing or bending of the distal increased-flexibility section 322 of the external elongate member 320 with flexing or bending or other manipulation or navigation of the increased-flexibility section 222 of the flexible tubular elongate member 220 of the medical scope 200, at least when the distal increased-flexibility section 322 is coupled with the increased-flexibility section 222. In some aspects, the segments 328 are more rigid than the cables 330, such as to impart a degree of rigidity to the overall structure of the distal increased-flexibility section 322 and/or to resist torsion of the distal increased-flexibility section 322. In some aspects, the segments 328 are generally solid, such as formed of an injection molded plastic (e.g., acrylonitrile butadiene styrene (ABS), etc.), that will not deform under the load that can be exerted by either the tissue or the forces created by articulation of the distal increased-flexibility section 322 of the external elongate member 320 or the increased-flexibility section 222 of the medical scope 200. The cables 330 may be able to slide within the apertures 331 in the segments 328 a sufficient amount to allow for potential expansion of the cables 330 resulting from bending of the distal increased-flexibility section 322. A degree of friction between the segments 328 and cables 330 is selected so that relative movement of the segments 328 and cables 330 occurs only when a bending force is applied (e.g., by the increased-flexibility section 222 of the flexible tubular elongate member 220 of the medical scope 200) which cannot cause bending of the distal increased-flexibility section 322 without sliding of the cables 330 relative to the segments 328. In some aspects, the segments 328 are returned to their initial spacing when the distal increased-flexibility section 322 is returned to its initial position/configuration. In some aspects, friction in the system should be high enough to allow for lift of the tissue, such as approximately twice the tissue weight. In some aspects, the cables 330 may be locked at a selected length for holding the position/configuration of the distal increased-flexibility section 322. It will be appreciated that the distalmost segment 228d may be fixed with respect to the cables 330, such as for the sake of structural stability. The configuration of the distal increased-flexibility section 322 of the external elongate member 320 as illustrated in FIGS. 1-3 with segments 328 and cables 330 provides sufficient structural rigidity to resist torsion as the distal end 100d of the system 100 (including the distal end 200d of the medical scope 200 and the distal end 300d of the auxiliary elongate member 300) is navigated within a patient's body, as well as torsion as the increased-flexibility section 222 of the flexible tubular elongate member 220 of the medical scope 200 is manipulated.

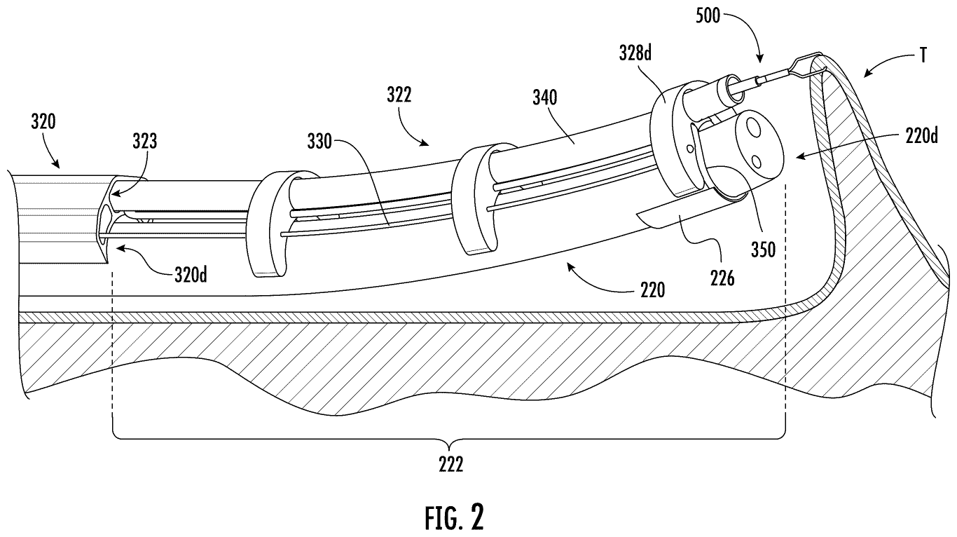

As noted above, the auxiliary elongate member 300 is configured to deliver a medical tool to a treatment site within a patient's body in addition to a tool delivered by the medical scope 200. In some aspects, the above-described tool lumen 321 of the external elongate member 320 of the auxiliary elongate member 300 is defined by a lumen 323 extending longitudinally through the proximal portion 324 of the external elongate member 320 from the proximal end 324p to the distal end 324d of the proximal portion 324. The tool lumen 321 may further be defined by tool apertures 325 defined through the segments 328 of the distal increased-flexibility section 322. In some aspects, a tool 500 is extended directly through the tool lumen 321 and the tool apertures 325. In some aspects, a flexible tubular elongate member 340 is provided through the tool lumen 321 and the tool apertures 325 (such as illustrated in FIG. 2 and FIG. 3) and defines a tool lumen 341 through which the tool 500 is axially translatable. In some aspects, the segments 328 are attached to the flexible tubular elongate member 340, and the flexibility of the flexible tubular elongate member 340 allows the flexible tubular elongate member 340 to compress/extend to compensate for any required movement of the segments 328. Preferably the tool apertures 325 are axially and radially oriented with respect to one another so that a tubular elongate member 340 and/or a tool 500 may readily extend therethrough.

In some aspects, it may be desirable for the distal end 300d of the auxiliary elongate member 300 to remain close to (adjacent, and optionally contacting) the distal end 200d of the medical scope 200, such as during insertion (e.g., intubation) into the patient, during navigation through a patient's body (e.g., through curved and/or tortuous body passages), and/or to maintain visibility of the distal end 300d of the auxiliary elongate member 300 with a viewing device on the distal end 200d of the medical scope 200. In accordance with various principles of the present disclosure, the relative positions of the distal increased-flexibility section 322 of the external elongate member 320 and the increased-flexibility section 222 of the flexible tubular elongate member 220 may be determined by a return element 350. The return element 350 may be an elongate flexible element, such as, without limitation, a filament, a string, a cord, a wire, a tether, etc., capable of maintaining the distal increased-flexibility section 322 in proximity with the increased-flexibility section 222. The return element 350 is also movable to allow adjustment of the relative positions of the distal increased-flexibility section 322 and the increased-flexibility section 222, as described in further detail below.

The return element 350 is illustrated in FIG. 1 as extending through a port 314 in the control handle 310 of the auxiliary elongate member 300 to extend distally through the auxiliary elongate member 300 to the distal end 300d of the auxiliary elongate member 300 to be coupled with the distal end 220d of the flexible tubular elongate member 220 of the medical scope 200. The return element 350 may extend through the tool lumen 323 defined through the proximal portion 324 of the external elongate member 320 (or a separate lumen extending axially through the proximal portion 324) and through the return element apertures 351 defined through the segments 328 to a distal end 350d. The distal end 350d of the return element 350 extends from the distal increased-flexibility section 322 of the external elongate member 320 to the distal end 220d of the flexible tubular elongate member 220. In some aspects, the distal end 350d of the return element 350 loops around the distal end 220d of the flexible tubular elongate member 220. In some aspects, the distal end 350d of the return element 350 is fixed to the exterior of the distal end 220d of the flexible tubular elongate member 220. In some aspects, a mount 226 is provided along the distal end 220d of the flexible tubular elongate member 220, and the distal end 350d of the return element 350 is operably coupled to the distal end 220d of the flexible tubular elongate member 220 via the mount 226. In some aspects, the mount 226 is fixedly coupled to the distal end 220d of the flexible tubular elongate member 220, and may extend partially or fully around the circumference of the distal end 220d of the flexible tubular elongate member 220. Various other configurations are within the scope of the present disclosure. For instance, the return element 350 may extend through the working channel 221 of the medical scope 200 and be fixed to a distal end 300d of the auxiliary elongate member 300.

As illustrated schematically in FIG. 2, the flexible tubular elongate member 220 and the external elongate member 320 are deliverable to a treatment site T within a patient's body to each deliver a tool 400, 500, respectively, configured to be used to perform a procedure with respect to the treatment site. A medical professional may hold the proximal end 350p of the return element 350 (e.g., in a fixed position and/or in tension), such as by holding a grasping handle 352 (e.g., an enlarged grasping element,, a knob, a lever, etc., provided along the proximal end 350p of the return element 350 such as illustrated in FIG. 1) configured to facilitate grasping and/or manipulation of the return element 350. The grasping handle 352 may be maintained in a coupling position maintaining the distal increased-flexibility section 322 of the external elongate member 320 in a position adjacent the increased-flexibility section 222 of the flexible tubular elongate member 220. Additionally or alternatively, the proximal end 350p of the return element 350 may be mounted on or otherwise coupled to the control handle 310 (e.g., wrapped around a post 354, or otherwise controlled by a knob, lever, etc., such as in a manner known to those of ordinary skill in the art) to maintain a selected position or length of the return element 350, and thus to maintain a desired position of the distal increased-flexibility section 322 of the external elongate member 320 relative to the increased-flexibility section 222 of the flexible tubular elongate member 220. As may be appreciated, the tool 500 delivered by the external elongate member 320 may be used to grasp target tissue T and the flexible tubular elongate member 220 may be flexed upwardly to lift the external elongate member 320 coupled thereto to lift the target tissue T. In some aspects, the tool 500 may grasp target tissue T and then the increased-flexibility section 222 of the flexible tubular elongate member 220 may be flexed to lift the target tissue T away from surrounding tissue. In some aspects, the flexing of the increased-flexibility section 222 causes flexing of the tool 500 to allow the tool 500 to continue grasping the target tissue T, such as by extending proximally out of the flexible tubular elongate member 340 and flexing across the distal end 200d of the medical scope 200.

In accordance with various principles of the present disclosure, once the distal end 220d of the flexible tubular elongate member 220 and the distal end 320d of the external elongate member 320 are positioned within a patient's body (e.g., at or adjacent to a treatment site), such as with a tool 500 delivered by the external elongate member 320 grasping tissue, it may be desirable to move the increased-flexibility section 222 of the flexible tubular elongate member 220 independently of the distal increased-flexibility section 322 of the external elongate member 320. Because the external elongate member 320 may be a passive member of the system 100 which is not moved independently, but, instead, simply follows the movement of the flexible tubular elongate member 220, decoupling of the distal ends/sections of the external elongate member 320 and the flexible tubular elongate member 220 allows the increased-flexibility section 222 of the flexible tubular elongate member 220 to move independently of the distal increased-flexibility section 322 of the external elongate member 320. The return element 350 may be released (e.g., slackened or otherwise not held in tension) to allow separation of the increased-flexibility section 222 of the flexible tubular elongate member 220 from the distal increased-flexibility section 322 of the external elongate member 320. The distal increased-flexibility section 322 of the external elongate member 320 may thus be decoupled from a position immediately adjacent the increased-flexibility section 222 of the flexible tubular elongate member 220 and the distance between the flexible tubular elongate member 220 and the external elongate member 320 may be increased. The increased-flexibility section 222 of the flexible tubular elongate member 220 may be moved with minimal to no effect on the distal increased-flexibility section 322 of the external elongate member 320 once these sections 222, 322 are decoupled from each other. For instance, an increase in the length of the return element 340 between the increased-flexibility section 222 and the distal increased-flexibility section 322 allows the increased-flexibility section 222 to be moved (e.g., using the control knobs 212 of the control handle 210) relative to and/or away from the distal increased-flexibility section 322. The tool 400 delivered by the flexible tubular elongate member 220 may thus be moved apart from the tool 500 delivered by the external elongate member 320. As may be appreciated, in some aspects, decoupling of the distal increased-flexibility section 322 of the external elongate member 320 increases the ability of the separate tools 400, 500 delivered, respectively, by the external elongate member 320 and the flexible tubular elongate member 220 to be manipulated and/or steered and/or moved independently of one another, such as illustrated in FIG. 3.

In some aspects, the distal increased-flexibility section 322 of the external elongate member 320 has sufficient stiffness to remain in a configuration and position corresponding to a configuration and position of the increased-flexibility section 222 of the flexible tubular elongate member 220. Thus, upon decoupling of the increased-flexibility section 222 of the flexible tubular elongate member 220 from the distal increased-flexibility section 322 of the external elongate member 320, the distal increased-flexibility section 322 remains in the position of the increased-flexibility section 222 of the flexible tubular elongate member 220 prior to decoupling, whereas the decoupled increased-flexibility section 222 of the flexible tubular elongate member 220 is movable into another configuration and/or position. In some aspects, the one or more cables 330 of the external elongate member 320 impart the distal increased-flexibility section 322 of the external elongate member 320 with sufficient stiffness to remain in a given position unless actively moved by an external force, such as by movement of the increased-flexibility section 222 of the flexible tubular elongate member 220. Additionally or alternatively, the cables 330 may be locked in place (in any of a variety of manners known to those of ordinary skill in the art) to maintain a desired length/position of the cables 330. Additionally or alternatively, in some aspects, a flexible tubular elongate member 340 (if provided) and/or a shaft of a tool 500 extending along or through the distal increased-flexibility section 322 of the external elongate member 320 imparts the distal increased-flexibility section 322 of the external elongate member 320 with sufficient stiffness to remain in a given position unless actively moved by an external force. In some aspects, the tool 500 delivered by the auxiliary elongate member 300 may be engaged with tissue at the treatment site T, such as illustrated in FIG. 2 and FIG. 3, and such engagement holds the distal increased-flexibility section 322 in place as the increased-flexibility section 222 is moved away. In some aspects, the tool 500 has sufficient stiffness to hold the configuration of the distal increased-flexibility section 322 of the external elongate member 320 set by the increased-flexibility section 222 of the flexible tubular elongate member 220, such as illustrated in FIG. 3, even if the tool 500 may be flexed to grasp onto target tissue T.

As may be appreciated, after separation of the increased-flexibility sections 222, 322, it may be desirable for the increased-flexibility sections 222, 322 to be returned to positions adjacent each other. The return element 350 is longitudinally translatable, as noted above, and may be operated, such as pulled proximally, to return the distal increased-flexibility section 322 of the external elongate member 320 toward the increased-flexibility section 222 of the flexible tubular elongate member 220. As may be appreciated, the return element 350 thus provides a return-to-home function to move the otherwise passive distal increased-flexibility section 322 of the external elongate member 320. It will be appreciated that such homing feature may be used more generally to adjust the distance between the distal increased-flexibility section 322 and the increased-flexibility section 222, and is not limited to returning the distal end 320d of the external elongate member 320 to a position adjacent to (e.g., in contact with) the distal end 220d of the flexible tubular elongate member 220.

As noted above, at least a portion of the external elongate member 320 of the auxiliary elongate member 300 may be shaped to complement the shape of the exterior of the flexible tubular elongate member 220 of the medical scope 200. In some aspects, at least the distalmost segment 328d may be sized, shaped, configured, and/or dimensioned to complement the shape of the exterior of the increased-flexibility section 222 of the flexible tubular elongate member 220, as may be appreciated with reference to FIGS. 1-3. In some aspects, the distalmost segment 328d may be sized, shaped, configured, and/or dimensioned to cradle the increased-flexibility section 222 of the flexible tubular elongate member 220, such as to guide the distalmost segment 328d into position adjacent (and, optionally, in contact with) the increased-flexibility section 222 of the flexible tubular elongate member 220. Additionally or alternatively, at least the distalmost segment 328d may be sized, shaped, configured, and/or dimensioned to hold the distal increased-flexibility section 322 of the external elongate member 320 in place laterally with respect to the increased-flexibility section 222 of the flexible tubular elongate member 220. As noted above, the segments 328 may also be sized, shaped, configured, and/or dimensioned to inhibit or prevent torsional loading on the distal increased-flexibility section 322 as the increased-flexibility section 222 of the flexible tubular elongate member 220 moves the distal increased-flexibility section 322 of the external elongate member 320. Additionally or alternatively, the location of the cables 330 with respect to the segments 328 may facilitate orienting of the distal increased-flexibility section 322 with respect to the increased-flexibility section 222 and/or may contribute to resistance to rolling and/or twisting of the distal increased-flexibility section 322 as it is moved. As may be appreciated, various features of the distal increased-flexibility section 322 of the external elongate member 320 contribute to orientable and/or predictable and/or stable movement of the distal increased-flexibility section 322, such as to achieve mating engagement with the increased-flexibility section 222 of the flexible tubular elongate member 220.

Although a system 100 as illustrated in FIG. 1 is illustrated in FIG. 2 and FIG. 3 as allowing movement of a cutting device and a tissue grasper, it will be appreciated that the auxiliary elongate member 300 may be used to deliver instruments other than a tissue grasper independently of an instrument (not limited to a cutting device) delivered by a medical scope 200 or other primary delivery device. Moreover, the instrument delivered by the medical scope 200, or other primary delivery device, may be moved independently of the instrument delivered by the auxiliary elongate member 300 without affecting the movement of the instrument delivered by the auxiliary elongate member 300.

It will be appreciated that embodiments of the present disclosure may be to treat tissues at various locations within a patient's body, such as, without limitation, the gastrointestinal system, the abdominal cavity, the digestive system, the urinary tract, the reproductive tract, the respiratory system, the cardiovascular system, the circulatory system, etc. Various further benefits of the various aspects, features, components, and structures of a device and system, and associated methods, such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for delivering medical instruments into a patient's body, said system comprising:
a primary elongate member having a flexible tubular elongate member with a proximal portion proximal to a transition point along the flexible tubular elongate member, and an increased-flexibility section distal to the transition point and more flexible than the proximal portion; and
an auxiliary elongate member having an external elongate member with a proximal portion, and a distal increased-flexibility section extending distally from said proximal portion;
wherein:
said auxiliary elongate member is configured to extend along said primary elongate member and to be coupled adjacent to said primary elongate member to be moved by said primary elongate member; and
said distal increased flexibility section of said auxiliary elongate member is configured to be operably coupled with and to move with the increased-flexibility section of said primary elongate member and has sufficient structural stability to remain in a position determined by said primary elongate member before said primary elongate member is moved away from said auxiliary elongate member.

2. The system of claim 1, further comprising a return element coupling said primary elongate member and said auxiliary elongate member.

3. The system of claim 2, wherein said return element movably couples said distal increased flexibility section of said auxiliary elongate member with respect to said increased flexibility section of said primary elongate member.

4. The system of claim 3, wherein said return element is translatable to adjust the relative positions of said distal increased flexibility section of said auxiliary elongate member and said increased flexibility section of said primary elongate member.

5. The system of claim 1, wherein said proximal portion of said auxiliary elongate member has a distal end coupled to said flexible tubular elongate member of said primary elongate member along the transition point.

6. The system of claim 1, wherein said proximal portion of said auxiliary elongate member is a sheath configured to receive an exterior of said primary elongate member.

7. The system of claim 6, wherein said sheath defines a lumen for longitudinal passage of a medical instrument therethrough.

8. The system of claim 7, further comprising a flexible tubular elongate member extending through the lumen of said sheath and through said distal increased-flexibility portion of said auxiliary elongate member, and defining a tool lumen for longitudinal passage of a medical instrument therethrough.

9. The system of claim 1, wherein said distal increased-flexibility section of said auxiliary elongate member is formed with a plurality of spaced apart segments.

10. The system of claim 9, wherein said plurality of spaced apart segments are coupled together with control wires imparting structural stability to said distal increased-flexibility section.

11. A system for delivering medical instruments into a patient's body, said system comprising:
a flexible external elongate member having a proximal portion and a distal increased-flexibility section extending distally from said proximal portion; and
a medical instrument longitudinally translatable through a lumen defined through said flexible external elongate member;
wherein:
said proximal portion of said flexible external elongate member is configured to be coupled with a proximal portion of a flexible steerable elongate member proximal to a transition point along the flexible steerable elongate member;
said distal increased-flexibility section of said flexible external elongate member is configured to be operably coupled with and to move with a distal increased-flexibility section of a flexible steerable elongate member defined distal to the transition point; and
said medical instrument maintains a position of said distal increased-flexibility section of said flexible external elongate member determined by the flexible steerable elongate member before the increased-flexibility section of the flexible steerable elongate member is moved away from said distal increased-flexibility section of said flexible external elongate member.

18. The system of claim 11, further comprising a return element configured to couple said flexible external elongate member with the primary elongate member.

19. The system of claim 12, wherein said return element movably couples said distal increased flexibility section of said flexible external elongate member with respect to the increased flexibility section of the primary elongate member.

20. The system of claim 13, wherein said return element is controllable to adjust the relative positions of said distal increased flexibility section of said flexible external elongate member and the increased flexibility section of the primary elongate member.

21. The system of claim 11, further comprising a flexible tubular elongate member extending through said flexible external elongate member and defining a tool lumen for longitudinal passage of said medical instrument therethrough.

22. A method of delivering and operating separate medical instruments, said method comprising:
delivering a first medical instrument with a primary elongate member;
delivering a second medical instrument with an auxiliary elongate member extending along the primary elongate member;
manipulating a flexible distal end of the primary elongate member, including causing a flexible distal end of the auxiliary elongate member to be similarly manipulated; and
separating the flexible distal end of the primary elongate member from the flexible distal end of the auxiliary elongate member to manipulate the flexible distal end of the primary elongate member without also manipulating the flexible distal end of the auxiliary elongate member.

27. The method of claim 16, wherein separating the flexible distal end of the primary elongate member from the flexible distal end of the auxiliary elongate member leaves the flexible distal end of the auxiliary elongate member in the configuration of the primary elongate member prior to separation from the auxiliary elongate member.

28. The method of claim 16, further comprising returning the flexible distal end of the primary elongate member to a position adjacent the flexible distal end of the auxiliary elongate member.

29. The method of claim 16, further comprising controlling the relative positions of the flexible distal end of the primary elongate member and the flexible distal end of the auxiliary elongate member by longitudinally translating a return element coupled therebetween.

30. The method of claim 16, further comprising manipulating the return element to return the flexible distal end of the primary elongate member to a position adjacent the flexible distal end of the auxiliary elongate member.
